(19) United States
(12) Patent Application Publication
(10) Pub. No.: US 2020/0261479 A1
(43) Pub. Date: Aug. 20, 2020

(54) PROCESSES FOR THE PREPARATION OF AMORPHOUS TENOFOVIR ALAFENAMIDE HEMIFUMARATE AND A PREMIX THEREOF

(71) Applicant: MYLAN LABORATORIES LIMITED, Maharashtra (IN)

(72) Inventors: Ramakoteswara Rao Jetti, Maharashtra (IN); Amit Singh, Maharashtra (IN); Neelima Bhagavatula, Maharashtra (IN); Anjaneyaraju Indukuri, Maharashtra (IN); B. A. Ramireddy, Maharashtra (IN)

(73) Assignee: MYLAN LABORATORIES LIMITED, Maharashtra (IN)

(21) Appl. No.: 16/718,342

(22) Filed: Dec. 18, 2019

Related U.S. Application Data
(62) Division of application No. 15/541,157, filed on Jun. 30, 2017, now abandoned.

(30) Foreign Application Priority Data
Jan. 3, 2015 (IN) 14/MUM/2015

(51) Int. Cl. A61K 31/675; A61K 31/52; A61K 9/00; C07F 9/6561

(52) U.S. Cl. CPC A61K 31/675; C07F 9/65616; A61K 9/0053; A61K 31/52

(57) ABSTRACT

An amorphous form of tenofovir alafenamide hemifumarate and process for the preparation of the same. A premix of amorphous tenofovir alafenamide hemifumarate with pharmaceutically acceptable excipients and process for the preparation of the same are also disclosed.

### CROSS-REFERENCE TO RELATED APPLICATIONS

This application is a divisional of U.S. application Ser. No. 15/541,157, filed on Jun. 30, 2017 which was the U.S. national stage entry under 35 U.S.C. § 371 of PCT/IB2015/060068, filed on Dec. 31, 2015, which claims the benefit of Indian provisional patent application No. 14/MUM/2015, filed on Jan. 3, 2015, all of which are incorporated by reference in their entirety for all purposes.

### FIELD OF THE INVENTION

The present disclosure generally relates to the pharmaceutical arts, and to amorphous forms of tenofovir alafenamide hemifumarate and processes for preparing the same. The present disclosure further relates to premixes of amorphous tenofovir alafenamide hemifumarate and processes for the preparation of the same.

### BACKGROUND OF THE INVENTION

Tenofovir alafenamide is chemically known as 9-{(R)-2-[((S)-{[(S)-1-(isopropoxycarbonyl) ethyl]amino} phenoxyphosphinyl)methoxy]propyl}adenine (as shown in Formula-I). Tenofovir alafenamide is a prodrug of tenofovir and may be useful alone or in combination with one or more anti-retroviral drugs for treating HIV infections as well as chronic hepatitis B.

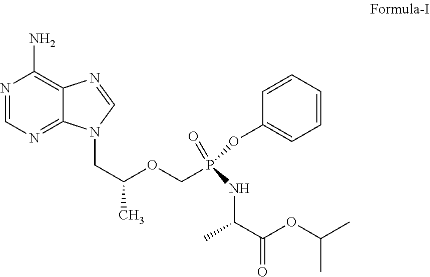 [SMILES: CC(C)OC(=O)[C@H](C)N[P@](=O)(CO[C@H](C)Cn1cnc2c(N)ncnc21)Oc1ccccc1]

Tenofovir alafenamide may come in a variety of salt forms, for example, tenofovir alafenamide hemifumarate, which is shown below in Formula-II.

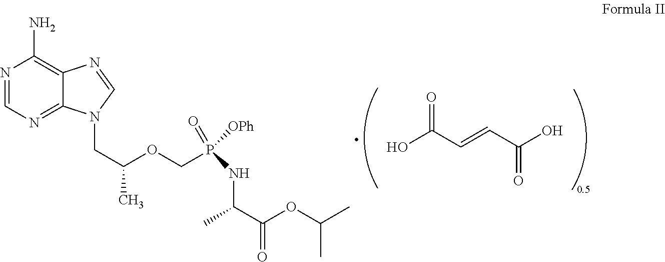

U.S. Pat. No. 7,390,791 discloses prodrugs of phosphonate nucleotide analogs, including tenofovir alafenamide and the fumarate salt thereof. U.S. Pat. No. 8,754,065 discloses tenofovir alafenamide hemifumarate in crystalline form.

Preparation of pharmaceutical dosage forms is often procedurally complex, particularly when combining the active ingredient with excipients. For example, workability or stability issues may arise when different components of the pharmaceutical dosage form come into intimate contact with one another. It may, thus, be advantageous to supply the manufacturer of pharmaceutical dosage forms with a pre-combined mixture (pre-mix) of excipients and active pharmaceutical ingredient (API) to facilitate and simplify the final processing of dosages forms.

### SUMMARY OF THE INVENTION

The present disclosure provides amorphous tenofovir alafenamide hemifumarate, as well as a process for preparing amorphous tenofovir alafenamide hemifumarate.

Within the context of the present invention, amorphous tenofovir alafenamide hemifumarate may be prepared by the steps of:
a) dissolving tenofovir alafenamide hemifumarate in a solvent to form a solution; and
b) removing the solvent to isolate the amorphous tenofovir alafenamide hemifumarate.

Another aspect of the present disclosure provides a premix of amorphous tenofovir alafenamide hemifumarate and process for the preparation of the same.

Within the context of the present invention, premixes of amorphous tenofovir alafenamide hemifumarate may be prepared by the steps of:
a) dissolving tenofovir alafenamide hemifumarate in a solvent to form a solution;
b) combining the solution with one or more pharmaceutically acceptable excipients; and
c) removing the solvent to isolate a premix of amorphous tenofovir alafenamide hemifumarate.

Within the context of the methods of generating premixes of amorphous tenofovir alafenamide hemifumarate, the pharmaceutically acceptable excipient may be dissolved in a second solvent to form a second solution. The first and the second solvents used in these methods may be the same or different.

The solvents used in the methods of the present invention may be selected from the group consisting of alcohol solvent, a ketone solvent, a chlorinated solvent, water, and miscible mixtures thereof.

The alcohol solvent may be selected from the group consisting of methanol, ethanol, propanol, isopropanol, n-butanol, sec-butanol, 2-butanol, t-butanol, 1-pentanol, 2-pentanol, 3-pentanol, 2-methyl-1-butanol, 2-methyl-1-butanol, 2,2-methyl-2-butanol, 3-methyl-2-butanol, 2,2-dimethyl-1-propanol, 1,1-dimethyl-1-propanol, and mixtures thereof.

The ketone solvent may be selected from the group consisting of acetone, methylethyl ketone, methylisobutyl ketone, 2-butanone, and mixtures thereof.

The chlorinated solvent may be selected from the group consisting of dichloromethane, 1,1-dichloroethane, 1,2-dichloroethane, chloroform, carbon tetrachloride, and mixtures thereof.

The solvent used in the preparation of amorphous tenofovir alafenamide hemifumarate or premixes of amorphous tenofovir alafenamide hemifumarate may be removed by evaporation, distillation, spray drying, lyophilization, or agitated thin film drying.

The pharmaceutically acceptable excipient used in the generation of premixes of amorphous tenofovir alafenamide hemifumarate may be selected from the group consisting of polysaccharides, polyvinylpyrrolidone, polyvinyl acetate, polyvinyl alcohol, polymers of acrylic acid and salts thereof, polyacrylamide, polymethacrylates, vinylpyrrolidone-vinyl acetate copolymers, C₁-C₆ polyalkylene glycols, and mixtures thereof.

Examples of suitable polysaccharides include hydroxypropyl methyl cellulose, croscarmellose, carboxymethyl cellulose, a sodium salt of carboxymethyl cellulose, a calcium salt of carboxymethyl cellulose, methyl cellulose, hydroxyethyl cellulose, ethyl hydroxyethyl cellulose, hydroxypropyl cellulose, microcrystalline cellulose, optionally substituted α-cyclodextrins, optionally substituted β-cyclodextrins, optionally substituted γ-cyclodextrins, and mixtures thereof.

An example of a suitable polyvinylpyrrolidine includes povidone with a K-value of about 30.

An example of a suitable vinylpyrrolidone-vinyl acetate copolymer includes N-vinyl-2-pyrrolidone and vinyl acetate in a 60:40 ratio, by mass. Examples of optionally substituted β-cyclodextrins include β-cyclodextrin, hydroxypropyl-β-cyclodextrin, and mixtures thereof.

Examples of a suitable C₁-C₆ polyalkylene glycol include polyethylene glycol, polypropylene glycol, and mixtures thereof.

The premixes containing amorphous tenofovir alafenamide hemifumarate of the present invention are exceptionally stable. For example, the premixes containing amorphous tenofovir alafenamide hemifumarate of the present invention degrade less than about 1% when the premix is stored for three to six months at 5±3° C. and degrade less than about 1% when the premix is stored for three to six months at 25° C. and at 60% relative humidity.

The premixes containing amorphous tenofovir alafenamide hemifumarate of the present invention may be used in the formulation of oral pharmaceutical dosage forms. These oral pharmaceutical dosage forms may include additional pharmaceutically acceptable excipients and additional active pharmaceutical ingredients. The additional active pharmaceutical ingredients may be selected from cobicistat, emtricitabine, elvitegravir, dolutegravir, lamivudine, nevirapine, efavirenz, atazanavir, ritonavir, nevirapine, rilpivirine, etravirine, darunavir, and pharmaceutically acceptable salts thereof. In certain embodiments, the oral pharmaceutical dosage form containing premixes of amorphous tenofovir alafenamide hemifumarate also include elvitegravir, cobicistat, emtricitabine, and pharmaceutically acceptable salts thereof as active pharmaceutical ingredients.

### BRIEF DESCRIPTION OF THE DRAWINGS

Further aspects of the present disclosure together with additional features contributing thereto and advantages accruing there from will be apparent from the following description of embodiments of the disclosure which are shown in the accompanying drawing figures wherein:

FIG. 1 is a powder X-ray diffraction (PXRD) pattern of amorphous tenofovir alafenamide hemifumarate;

FIG. 2 is an X-ray powder diffraction (PXRD) pattern of a premix of amorphous tenofovir alafenamide hemifumarate with 10% w/w povidone K-30;

FIG. 3 is an X-ray powder diffraction (PXRD) pattern of a premix of amorphous tenofovir alafenamide hemifumarate with 10% w/w hydroxypropyl-β-cyclodextrin;

FIG. 4 is an X-ray powder diffraction (PXRD) pattern of a premix of amorphous tenofovir alafenamide hemifumarate with 10% w/w β-cyclodextrin;

FIG. 5 is an X-ray powder diffraction (PXRD) pattern of a premix of amorphous tenofovir alafenamide hemifumarate with 10% w/w PLASDONE S-630;

FIG. 6 is an X-ray powder diffraction (PXRD) pattern of a premix of amorphous tenofovir alafenamide hemifumarate with 25% w/w PLASDONE S-630; and

FIG. 7 is an X-ray powder diffraction (PXRD) pattern of a premix of amorphous tenofovir alafenamide hemifumarate with 50% w/w PLASDONE S-630.

### DETAILED DESCRIPTION OF THE INVENTION

In one aspect, the present disclosure provides an amorphous form of tenofovir alafenamide hemifumarate and process for the preparation of the same.

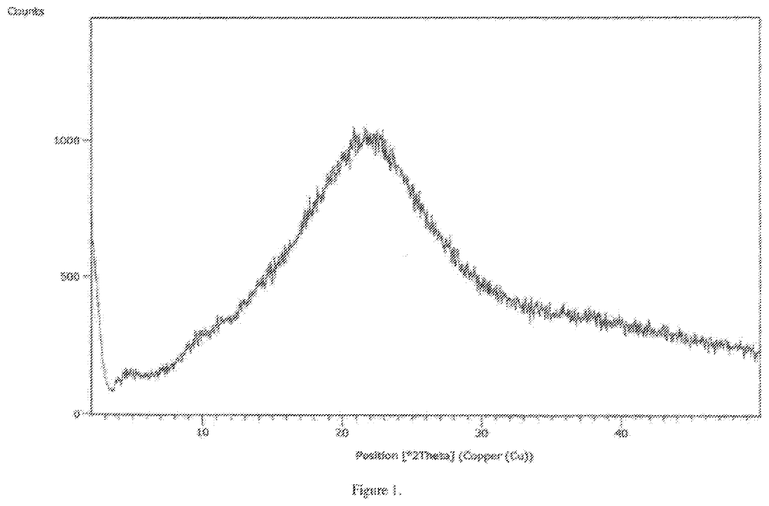

The amorphous form of tenofovir alafenamide hemifumarate of the present disclosure may be characterized by a PXRD pattern. Thus, the PXRD patterns of the amorphous form of tenofovir alafenamide hemifumarate were obtained on BRUKER D-8 Discover powder diffractometer equipped with goniometer of θ/2θ configuration and Lynx Eye detector. The Cu-anode X-ray tube was operated at 40 kV and 30 mA. The experiments were conducted over the 2θ range of 2.0°-50.0°, 0.030° step size and 0.4 seconds step time. According to one embodiment of the present disclosure, the amorphous form of tenofovir alafenamide hemifumarate possesses PXRD pattern as depicted in FIG. 1.

Another aspect of the present disclosure provides a process for the preparation of amorphous tenofovir alafenamide hemifumarate, comprising:
a) dissolving tenofovir alafenamide hemifumarate in a solvent to form a solution;
b) removing the solvent to isolate the amorphous tenofovir alafenamide hemifumarate.

According to the present disclosure, tenofovir alafenamide hemifumarate may be dissolved in a solvent to form a solution. Within the context of the present disclosure, the tenofovir alafenamide hemifumarate starting material may be any polymorphic or amorphous.

Within the context of the present disclosure, the solvent may be, for example, an alcohol solvent, a ketone solvent, a chlorinated solvent, water, or a miscible mixture thereof.

Examples of suitable alcohol solvents include methanol, ethanol, propanol, isopropanol, n-butanol, sec-butanol, 2-butanol, t-butanol, 1-pentanol, 2-pentanol, 3-pentanol, 2-methyl-1-butanol, 2-methyl-1-butanol, 2-2-methyl-2-butanol, 3-methyl-2-butanol, 2,2-dimethyl-1-propanol, 1,1,dimethyl-1-propanol, and mixtures thereof.

Examples of suitable ketone solvents include acetone, methylethyl ketone, methylisobutyl ketone, 2-butanone, and mixtures thereof. Examples of suitable chlorinated solvents include dichloromethane, 1,1-dichloroethane, 1,2-dichloroethane, chloroform, carbon tetrachloride, and mixtures thereof.

In some embodiments of the present disclosure, dissolving tenofovir alafenamide hemifumarate in an alcohol solvent was found to be useful. In some embodiments of the present disclosure, dissolving tenofovir alafenamide hemifumarate in methanol was found to be particularly useful.

According to the present disclosure, the solvent may then be removed from the solution to isolate amorphous tenofovir alafenamide hemifumarate. This may be carried out by well-known techniques such as, for example, evaporation, distillation, spray drying, lyophilization, agitated thin film drying, or combinations thereof. In certain embodiments of the present disclosure, it was found that spray drying was particularly useful for removing the solvent.

The amorphous tenofovir alafenamide hemifumarate of the present disclosure possesses several benefits. For example, amorphous tenofovir alafenamide hemifumarate is particularly stable and, as describe more fully below, may be easily formulated into a premix useful in generating pharmaceutical formulations. Additionally, the amorphous tenofovir alafenamide hemifumarate described herein may possess improved workability (e.g., flowability, tackiness), and may permit the use of formulation techniques, such as dry and/or wet granulation with one or more additional pharmaceutically acceptable excipients as described below.

Within the context of the present disclosure, the amorphous form of tenofovir alafenamide hemifumarate of the present disclosure may exhibit long-term physical and chemical stability. As an example, Table 1 below shows data collected on amorphous tenofovir alafenamide hemifumarate prepared the processes disclosed hereinabove. The amorphous tenofovir alafenamide hemifumarate tested shows no significant degradation or change in PXRD pattern (e.g., is stable at 3 and 6 months storage) when stored at 5±3° C. and at 25° C./60% relative humidity (RH).

As used herein, a compound or pharmaceutical composition is considered “stable” where the HPLC purity of the compound or premix changes by less than about 1% when stored at 5±3° C. and/or at 25° C./60% relative humidity (RH). In certain embodiments, the “stable” compound or premix is stored at 5±3° C. In other embodiments, the “stable” compound or premix is stored at 25° C./60% relative humidity (RH).

TABLE 1

| | Amorphous tenofovir alafenamide hemifumarate | |
|---|---|---|
| Condition | HPLC purity (%) | PXRD |
| at 25° C./60% RH | | |
| Initial | 99.53 | Amorphous |
| 3 months | 99.36 | Stable |
| 6 months | 99.48 | Stable |
| at 5 ± 3° C. | | |
| Initial | 99.53 | Amorphous |
| 3 months | 99.45 | Stable |
| 6 months | 99.54 | Stable |

In another aspect, the present disclosure provides premixes containing amorphous tenofovir alafenamide hemifumarate and process for the preparation of the same. In certain embodiments, the premix of amorphous tenofovir alafenamide hemifumarate may include, for example, amorphous tenofovir alafenamide hemifumarate and one or more pharmaceutically acceptable excipients. In certain other embodiments, the premix of amorphous tenofovir alafenamide hemifumarate consist essentially of, or consist of, amorphous tenofovir alafenamide hemifumarate and one or more pharmaceutically acceptable excipients.

Another aspect of the present disclosure provides a method for preparing a premix of amorphous tenofovir alafenamide hemifumarate, comprising:
a) dissolving tenofovir alafenamide hemifumarate in a solvent to form a solution;
b) combining the solution with one or more pharmaceutically acceptable excipients; and
c) removing the solvent to isolate a premix of amorphous tenofovir alafenamide hemifumarate.

According to this aspect of the present disclosure, tenofovir alafenamide hemifumarate may be dissolved in a solvent to form a solution. The tenofovir alafenamide hemifumarate starting material may be any polymorphic form or amorphous. Within the context of the present disclosure, the solvent may be, for example, the same or different as those listed above for use in the preparation of amorphous tenofovir alafenamide. Suitable examples of solvents include an alcohol solvent, a ketone solvent, a chlorinated solvent, water, and miscible mixtures thereof.

Examples of suitable alcohol solvents include methanol, ethanol, propanol, isopropanol, n-butanol, sec-butanol, 2-butanol, t-butanol, 1-pentanol, 2-pentanol, 3-pentanol, 2-methyl-1-butanol, 2-methyl-1-butanol, 2,2-methyl-2-butanol, 3-methyl-2-butanol, 2,2-dimethyl-1-propanol, 1,1-dimethyl-1-propanol, and mixtures thereof.

Examples of suitable ketone solvents include acetone, methylethyl ketone, methylisobutyl ketone, 2-butanone, and mixtures thereof. Examples of suitable chlorinated solvents include dichloromethane, 1,1-dichloroethane, 1,2-dichloroethane, chloroform, carbon tetrachloride, and mixtures thereof.

In some embodiments of the present disclosure, dissolving tenofovir alafenamide hemifumarate in an alcohol solvent was found to be particularly useful. In some embodiments of the present disclosure, dissolving tenofovir alafenamide hemifumarate in methanol was found to be particularly useful.

According to embodiments of the present disclosure, the solution may further comprise by one or more pharmaceutically acceptable excipients. Examples of suitable pharmaceutical excipients include, but are not limited to, polysaccharides, polyvinylpyrrolidone, polyvinyl acetate (PVAC), polyvinyl alcohol (PVA), polymers of acrylic acid and their salts, polyacrylamide, polymethacrylates, vinylpyrrolidone-vinyl acetate copolymers, C₁-C₆ polyalkylene glycols (e.g., polypropylene glycol, polyethylene glycol), copolymers of polyethylene glycol and polypropylene glycol (e.g., the families of block copolymers based on ethylene oxide and propylene oxide sold under the PLURONIC® tradename), and mixtures thereof. Suitable polysaccharides include, for example, microcrystalline cellulose, hydroxypropyl methylcellulose (HPMC), croscarmellose, carboxymethyl cellulose (CMC) and salts thereof, methyl cellulose, hydroxyethyl cellulose, ethyl hydroxyethyl cellulose, hydroxypropyl cellulose (HPC), optionally substituted α-cyclodextrins, optionally substituted β-cyclodextrins (e.g., hydroxypropyl β-cyclodextrin), optionally substituted γ-cyclodextrins (e.g., hydroxypropyl γ-cyclodextrin) and mixtures thereof. As used herein, the term “substituted” with respect to cyclodextrin means the addition of side chain groups such as hydroxyl, hydroxypropyl, and other C₁-C₆ alkyl and C₁-C₆ hydroxyalkyl.

In some embodiments, it was found that adding vinylpyrrolidone-vinyl acetate copolymer, polyvinylpyrrolidone, or a cyclodextrin (e.g., an optionally substituted α-cyclodextrin, an optionally substituted β-cyclodextrin, or an optionally substituted γ-cyclodextrin) to the solution of tenofovir permitted the generation of useful tenofovir premixes. Within the context of the present invention, povidone with K-values ranging from about 12 to about 103 may be useful, including povidone K-12, povidone K-15, povidone K-17, povidone K-25, povidone K-30, povidone K-90, and mixtures thereof. One of skill in the art would readily recognize different forms of povidone that would be useful and how each form may confer desired properties to the final dosage form.

In particularly useful embodiments of the present disclosure, a copolymer of N-vinyl-2-pyrrolidone and vinyl acetate may be utilized as the pharmaceutically acceptable excipients added to the tenofovir alafenamide hemifumarate solution. Suitable examples of copolymers include a copolymer of N-vinyl-2-pyrrolidone and vinyl acetate with a mass ratio of 60:40 (e.g., PLASDONE S-630 or KOLLIDON® VA 64).

In other embodiments, polyvinylpyrrolidone was found to be particularly useful as the pharmaceutically acceptable excipient. Polyvinylpyrrolidone with a K-value of about 30 and an average molecular weight of 40 kDa (i.e., povidone K-30) was found to be particularly useful as the pharmaceutically acceptable excipient. In yet other particularly useful embodiments of the present disclosure, β-cyclodextrin or hydroxypropyl-β-cyclodextrin may be employed as the pharmaceutically acceptable excipient. The pharmaceutically acceptable excipients may be combined with the tenofovir alafenamide hemifumarate solution either as solids or solutions in which the pharmaceutically acceptable excipients are dissolved. The solvent used to dissolve the pharmaceutically acceptable excipient and tenofovir alafenamide hemifumarate may be different from or the same as the solvent used for the generation of amorphous tenofovir alafenamide hemifumarate described above.

As used herein, the term “molecular weight” means the weight-averaged molecular weight (M_{w}).

Within the context of this embodiment of the present disclosure, the pharmaceutically acceptable excipient may be combined with the solution of tenofovir alafenamide hemifumarate from about 10% w/w (pharmaceutically acceptable excipient/total composition mass) to about 50% w/w, which may be about 10% w/w, 15% w/w, 20% w/w, 25% w/w, 30% w/w, 35% w/w, 40% w/w, 45% w/w, 50% w/w, or between any of the aforementioned w/w percentages, including the ranges of about 10%-40%, 10%-30%, 10%-20%, 20%-50%, 20%-40%, 20%-30%, 30%-50%, 30%-40%, and 40%-50% w/w. In some embodiments of the present disclosure, combining a vinylpyrrolidone-vinyl acetate copolymer (e.g., a copolymer with a 40:60 ratio of N-vinyl-2-pyrrolidone to vinyl acetate) at concentrations recited above, including from about 10% to 50% w/w, with tenofovir alafenamide hemifumarate was found to be useful. In other embodiments of the present disclosure, combining polyvinylpyrrolidone (e.g., a polyvinylpyrrolidone with a K-value of 30) with tenofovir alafenamide hemifumarate at concentrations recited above, including from about 10% to 50% w/w, was found to be useful. In yet other embodiments of the present disclosure, combining β-cyclodextrin with tenofovir alafenamide hemifumarate at concentrations recited above, including from about 10% to 50% w/w, was found to be useful. In further embodiments of the present disclosure, combining hydroxypropyl-β-cyclodextrin with tenofovir alafenamide hemifumarate at concentrations recited above, including from about 10% to 50% w/w, was found to be useful.

According to the present disclosure, the solvent may then be removed from the solution to isolate a premix of amorphous tenofovir alafenamide hemifumarate. This may be carried out by well-known techniques, for example, evaporation, distillation, spray drying, lyophilization, or agitated thin film drying, or combinations thereof. In certain embodiments of the present disclosure, the technique of spray drying is particularly useful for removing the solvent.

The amorphous tenofovir alafenamide hemifumarate premixes of the present disclosure possess several benefits useful for formulating tenofovir alafenamide hemifumarate. For example, the amorphous tenofovir alafenamide hemifumarate premixes are particularly stable and may possess superior pharmaceutical workability (e.g., tackiness, flowability) and may permit the use of formulation techniques, such as dry and/or wet granulation. As such, the amorphous tenofovir alafenamide hemifumarate premixes are easily utilized in the generation of pharmaceutical formulations. In using the amorphous tenofovir alafenamide hemifumarate premixes described herein, one of ordinary skill in the art is now able to generate stable formulations containing amorphous tenofovir alafenamide hemifumarate efficiently and effectively.

With all of the reactions and processes disclosed above, one of skill in the art will recognize that the reaction conditions (e.g., reaction time, temperature) may be adjusted to achieve appropriate yield without undertaking undue experimentation and without departing from the scope of the present disclosure.

When prepared according to methods disclosed herein, the premixes of amorphous tenofovir alafenamide hemifumarate may be characterized by PXRD.

Thus, the PXRD patterns of several embodiments of the present disclosure were obtained on BRUKER D-8 Discover powder diffractometer equipped with goniometer of θ/2θ configuration and Lynx Eye detector. The Cu-anode X-ray tube was operated at 40 kV and 30 mA. The experiments were conducted over the 2θ range of 2.0°-50.0°, 0.030° step size and 0.4 seconds step time.

Further, the stability of certain embodiments of the premix of amorphous tenofovir alafenamide hemifumarate can be measured by PXRD and HPLC. Thus, HPLC analyses were also performed. HPLC separations were performed on a Waters Alliance 2695 HPLC system with UV detector. Data was collected and analyzed with Empower chromatography software or equivalent. The separation was performed on an X-Bridge Phenyl, 250×4.6 mm, 5 μm column with a mobile phase flow rate of 0.7 mL/min and injection volume of 10 μL. Separations were performed by gradient elution with mobile 1.75 mM tetrabutylammonium bisulfate (pH 2.0) in 5% THF (mobile phase A) and 70:30 acetonitrile:methanol (mobile phase B) as specified in the table below.

### Gradient Program:

| Time (min) | Mobile phase-A (% ν/ν) | Mobile phase-B (% ν/ν) |
|---|---|---|
| 0 | 100 | 0 |
| 8 | 100 | 0 |
| 21 | 90 | 10 |
| 25 | 90 | 10 |
| 30 | 85 | 15 |
| 40 | 65 | 35 |
| 41 | 100 | 0 |
| 55 | 100 | 0 |

The column oven temp was 20° C. and UV detector was set to 260 nm. The purity of the premix was also measured by HPLC using similar conditions.

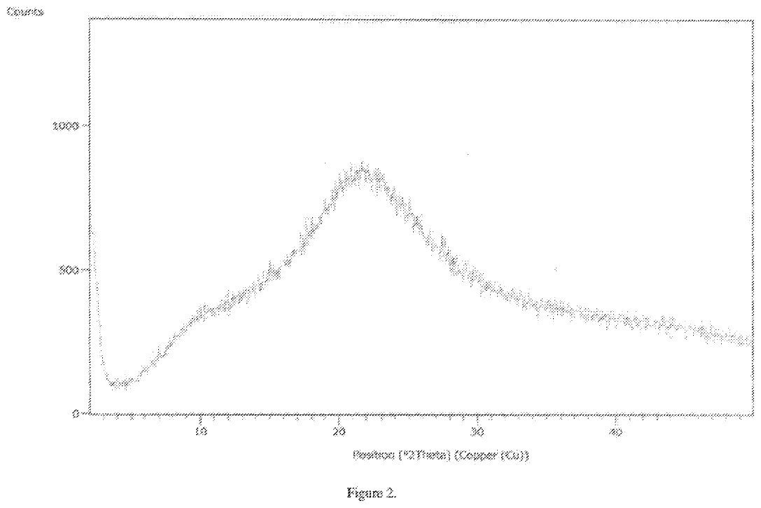

One embodiment of the present disclosure provides a premix of amorphous tenofovir alafenamide hemifumarate with 10% w/w povidone K-30, polyvinylpyrrolidone with an average molecular weight of 40 kDa. An example of a PXRD pattern of a premix of amorphous tenofovir alafenamide hemifumarate combined with 10% w/w povidone K-30 is shown in FIG. 2.

In some embodiments, the premix of amorphous tenofovir alafenamide hemifumarate with povidone K-30 prepared according to the present disclosure has an HPLC purity of greater than 99% (i.e., the premix contains 1% or less of impurities or components other than amorphous tenofovir alafenamide hemifumarate and povidone K-30).

Within the context of the present disclosure, the premix of amorphous tenofovir alafenamide hemifumarate may exhibit long-term physical and chemical stability. As an example, Table 2 below shows data collected on the premix of amorphous tenofovir alafenamide hemifumarate with 10% povidone K-30 prepared according to the processes disclosed in the present disclosure. According to data collected, the premix of amorphous tenofovir alafenamide hemifumarate prepared with 10% povidone K-30 tested shows no significant degradation or change in PXRD pattern (e.g., is stable at 3 and 6 months) when stored at 5±3° C. and at 25° C./60% relative humidity (RH). In certain particularly effective embodiments, the premix of amorphous tenofovir alafenamide hemifumarate and povidone displayed less than 1% degradation over six months under those conditions.

TABLE 2

| | Premix of amorphous tenofovir alafenamide | |
|---|---|---|
| | hemifumarate using 10% w/w povidone K-30 | |
| Condition | HPLC purity (%) | PXRD |
| at 25° C./60% RH | | |
| Initial | 99.31 | Premix amorphous |
| 2 months | 98.5 | Stable |
| 3 months | 98.9 | Stable |
| 6 months | 98.98 | Stable |
| at 5 ± 3° C. | | |
| Initial | 99.31 | Premix amorphous |
| 2 months | 98.5 | Stable |
| 3 months | 98.9 | Stable |
| 6 months | 98.99 | Stable |

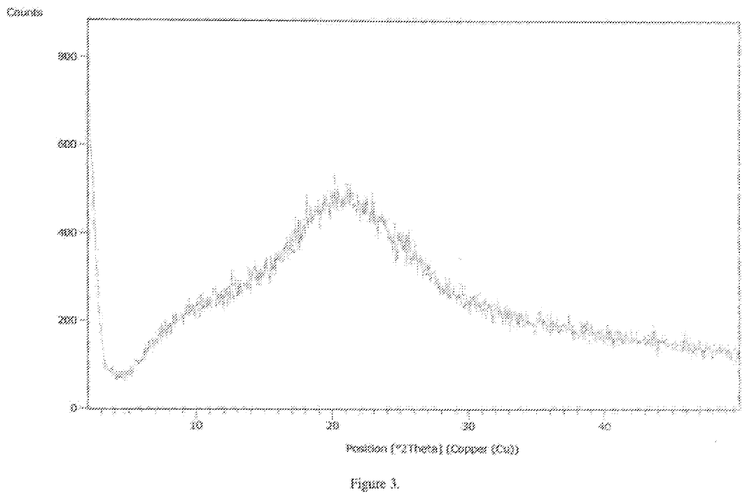

One embodiment of the present disclosure provides a premix of amorphous tenofovir alafenamide hemifumarate with 10% w/w hydroxypropyl-β-cyclodextrin (HPBCD). An example of a PXRD pattern of a premix of amorphous tenofovir alafenamide hemifumarate combined with 10% w/w HPBCD is shown in FIG. 3.

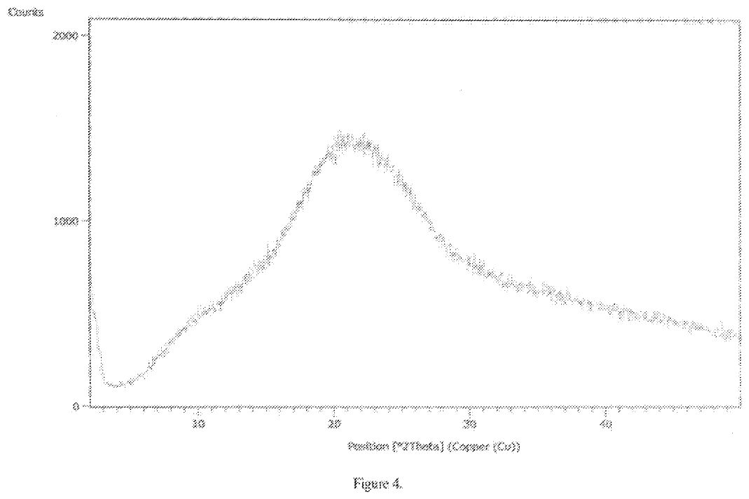

Another embodiment of the present disclosure provides a premix of amorphous tenofovir alafenamide hemifumarate with 10% w/w β-cyclodextrin (BCD). An example of a PXRD pattern of a premix of amorphous tenofovir alafenamide hemifumarate combined with 10% w/w BCD is shown in FIG. 4.

Premixes of amorphous tenofovir alafenamide hemifumarate that include HPBCD or BCD prepared according to the present disclosure may have HPLC purity of more than 99% (i.e., the premix contains 1% or less of impurities or components other than amorphous tenofovir alafenamide hemifumarate or HPBCD/BCD).

Within the context of the present disclosure, the premix of amorphous tenofovir alafenamide hemifumarate with HPBCD or BCD may exhibit long-term physical and chemical stability. As an example, Table 3 below shows data collected on the premix of amorphous tenofovir alafenamide hemifumarate with 10% w/w HPBCD as well as data collected on the premix of amorphous tenofovir alafenamide hemifumarate with 10% w/w BCD, both formulations prepared according to the processes disclosed in the present disclosure. The data demonstrate that the premixes of amorphous tenofovir alafenamide hemifumarate that include either 10% w/w HPBCD or 10% w/w BCD tested show no significant degradation or change in PXRD pattern (e.g., is stable at 3 and 6 months storage) when stored for up to 6 months at 5±3° C. or at 25° C./60% relative humidity (RH). In certain particularly effective embodiments, the premix of amorphous tenofovir alafenamide hemifumarate and a β-cyclodextrin (i.e., β-cyclodextrin or hydroxypropyl-β-cyclodextrin) displayed less than 1% degradation over six months under those conditions.

TABLE 3

| | 10% w/w HPBCD | | 10% w/w BCD | |
|---|---|---|---|---|
| | HPLC | | HPLC | |
| | purity | | purity | |
| Condition | (%) | PXRD | (%) | PXRD |
| at 25° C./60% RH | | | | |
| Initial | 99.58 | Premix | 99.45 | Premix |
| | | amorphous | | amorphous |
| 2 months | 99.35 | Stable | 99.3 | Stable |
| 3 months | 99.43 | Stable | 99.39 | Stable |
| 6 months | 99.49 | Stable | 99.5 | Stable |
| at 5 ± 3° C. | | | | |
| Initial | 99.58 | Premix | 99.45 | Premix |
| | | amorphous | | amorphous |
| 2 months | 99.35 | Stable | 99.29 | Stable |
| 3 months | 99.46 | Stable | 99.4 | Stable |
| 6 months | 99.59 | Stable | 99.51 | Stable |

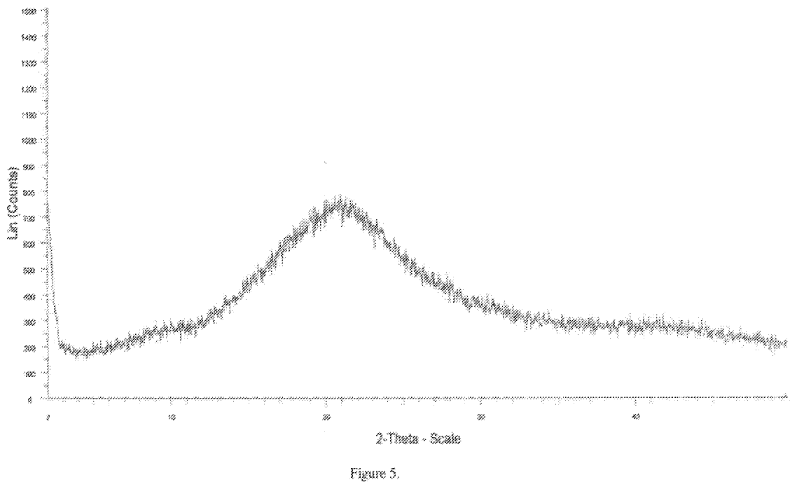

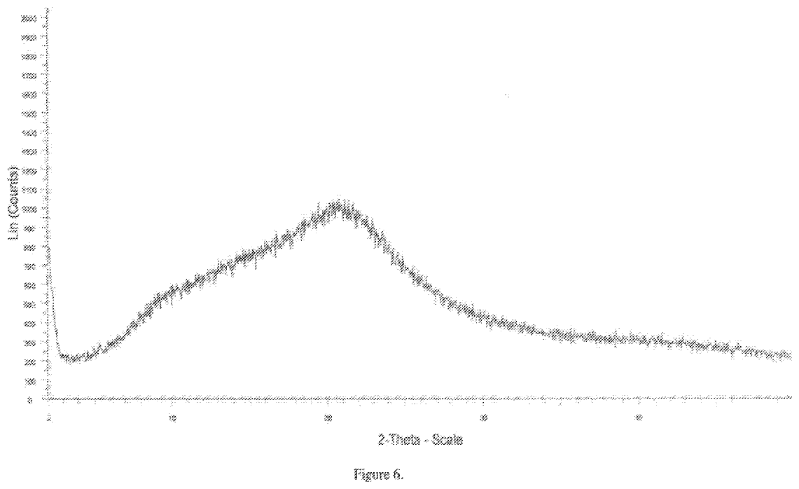

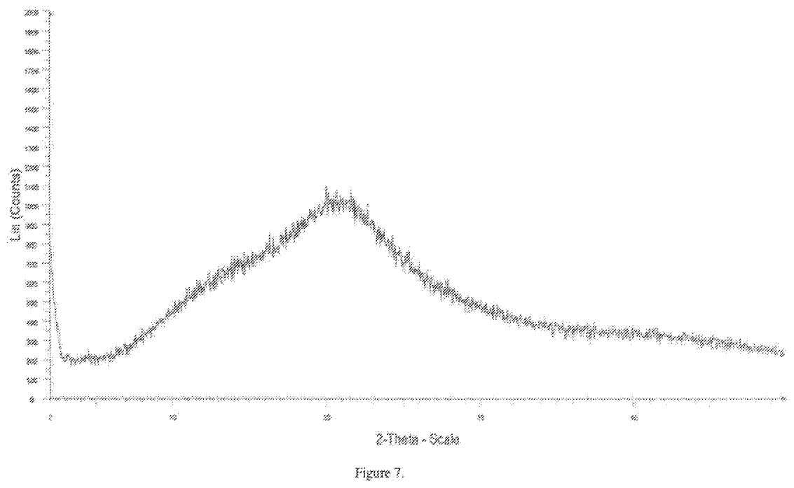

In another embodiment of the present disclosure provides a premix of amorphous tenofovir alafenamide hemifumarate with PLASDONE S-630 copovidone, which is a 60:40 (by mass) copolymer of N-vinyl-2-pyrrolidone and vinyl acetate. Examples of PXRD patterns of a premix of amorphous tenofovir alafenamide hemifumarate combined with 10% w/w, 25% w/w, or 50% w/w PLASDONE S-630 are shown in FIGS. 5, 6, and 7, respectively.

According to the present disclosure, a premix of amorphous tenofovir alafenamide hemifumarate that includes PLASDONE S-630 prepared according to the present disclosure may have HPLC purity of more than 99% (i.e., the premix contains 1% or less of impurities or components other than amorphous tenofovir alafenamide hemifumarate or PLASDONE S-630).

Within the context of the present disclosure, the premix of amorphous tenofovir alafenamide hemifumarate with PLASDONE S-630 may exhibit long-term physical and chemical stability. As an example, Table 4 below shows data collected on premixes of amorphous tenofovir alafenamide hemifumarate with 10% w/w PLASDONE S-630, 25% w/w PLASDONE S-630, and 50% w/w PLASDONE S-630, all prepared according to the processes disclosed in the present disclosure. The data demonstrate that the premixes of amorphous tenofovir alafenamide hemifumarate that include PLASDONE S-630 show no significant degradation or change in PXRD pattern (e.g., is stable at 3 and 6 months storage) when stored for 6 months at 5±3° C. or at 25° C./60% relative humidity (RH). In certain particularly effective embodiments, the premix of amorphous tenofovir alafenamide hemifumarate and PLASDONE S-630 displayed less than 1% degradation over six months under those conditions.

TABLE 4

| | 10% w/w | | 25% w/w | | 50% w/w | |
|---|---|---|---|---|---|---|
| | PLASDONE S-630 | | PLASDONE S-630 | | PLASDONE S-630 | |
| | HPLC | | HPLC | | HPLC | |
| | purity | | purity | | purity | |
| Condition | (%) | PXRD | (%) | PXRD | (%) | PXRD |

at 25° C./60% RH

| | | | | | | |
|---|---|---|---|---|---|---|
| Initial | 99.61 | Premix | 99.73 | Premix | 99.79 | Premix |
| | | amorphous | | amorphous | | amorphous |
| 3 months | 99.51 | Stable | 99.22 | Stable | 99.58 | Stable |
| 6 months | 99.51 | Stable | 99.26 | Stable | 99.63 | Stable |

at 5 ± 3° C.

| | | | | | | |
|---|---|---|---|---|---|---|
| Initial | 99.69 | Premix | 99.73 | Premix | 99.79 | Premix |
| | | amorphous | | amorphous | | amorphous |
| 3 months | 99.44 | Stable | 99.24 | Stable | 99.7 | Stable |
| 6 months | 99.51 | Stable | 99.34 | Stable | 99.7 | Stable |

The amorphous tenofovir alafenamide hemifumarate and premixes disclosed herein may be incorporated into oral pharmaceutical dosage forms, for example, a capsule or tablet.

Amorphous tenofovir alafenamide hemifumarate and premixes thereof may be combined singly or in combination with one or more additional active pharmaceutical ingredients or pharmaceutically acceptable salts thereof for the treatment of viral infections in subjects. In some formulations, amorphous tenofovir alafenamide hemifumarate may be effective as an anti-retroviral active pharmaceutical agent. Examples of active pharmaceutical ingredients with which amorphous tenofovir alafenamide hemifumarate may be combined includes cobicistat, emtricitabine, elvitegravir, dolutegravir, lamivudine, nevirapine, efavirenz, atazanavir, ritonavir, nevirapine, rilpivirine, etravirine, darunavir, and pharmaceutically acceptable salts thereof. The combination may be implemented as a single pharmaceutical dosage form or multiple pharmaceutical dosage forms. Suitable combinations of APIs include those listed in Table 5.

TABLE 5

Suitable combinations of APIs and amorphous tenofovir alafenamide
hemifumarate.
Atazanavir, ritonavir, lamivudine, and tenofovir alafenamide
hemifumarate
Cobicistat, darunavir ethanolate, emtricitabine, and amorphous
tenofovir alafenamide hemifumarate
Cobicistat, elvitegravir, emtricitabine, and amorphous tenofovir
alafenamide hemifumarate
Darunavir ethanolate , lamivudine, ritonavir, and amorphous
tenofovir alafenamide hemifumarate
Dolutegravir sodium, emtricitabine, and amorphous tenofovir
alafenamide hemifumarate
Dolutegravir sodium, lamivudine, and tenofovir alafenamide
hemifumarate
Efavirenz, emtricitabine, and tenofovir alafenamide hemifumarate
Efavirenz, lamivudine, and tenofovir alafenamide hemifumarate
Emtricitabine, rilpivirine hydrochloride, and tenofovir alafenamide
hemifumarate
Emtricitabine and tenofovir alafenamide hemifumarate
Emtricitabine, tenofovir alafenamide hemifumarate, and nevirapine
Etravirine, lamivudine, tenofovir alafenamide hemifumarate, and
etravirine
Lamivudine, rilpivirine hydrochloride, and tenofovir alafenamide
hemifumarate
Lamivudine and tenofovir alafenamide hemifumarate
Lamivudine, tenofovir alafenamide hemifumarate, and nevirapine

The oral dosage forms containing amorphous tenofovir alafenamide hemifumarate or premixes thereof may further comprise one or more additional pharmaceutically acceptable excipients, such as, but not limited to, croscarmellose sodium, hydroxypropyl cellulose, lactose monohydrate, magnesium stearate, microcrystalline cellulose, silicon dioxide, sodium lauryl sulfate, starch (including pregelatinized starch), povidone, polysorbate 20, as well as artificial colors and flavorings to prepare final dosage forms.

Capsules or tablets containing amorphous tenofovir alafenamide hemifumarate or premixes thereof as disclosed in herein may include a coating that contains, for example, artificial colorings and flavorings, polyethylene glycol, polyvinyl alcohol, talc, hypromellose, triacetin, lactose monohydrate, and titanium dioxide. One of skill in the art will recognize a variety of excipients that would be useful for creating suitable coatings for a final dosage form of tenofovir alafenamide hemifumarate. Within the context of the present disclosure, dosage forms containing amorphous tenofovir alafenamide hemifumarate may have between about 10 mg to about 300 mg per dose, including dosages of about 10 mg, 126 mg, 150 mg, 168 mg, 210 mg, 250 mg, 252 mg, 300 mg.

The pharmaceutical dosage forms containing amorphous tenofovir alafenamide hemifumarate or premixes thereof may be useful in treating HIV infection or hepatitis B.

In view of the above description and the examples below, one of ordinary skill in the art will be able to practice the disclosure as claimed without undue experimentation. The foregoing will be better understood with reference to the following examples that detail certain procedures for the preparation of molecules, compositions, and formulations according to the present disclosure. All references made to these examples are for the purposes of illustration. The following examples should not be considered exhaustive, but merely illustrative of only a few of the many aspects and embodiments contemplated by the present disclosure.

### EXAMPLES

### Example 1: Preparation of Amorphous Tenofovir Alafenamide Hemifumarate

Tenofovir alafenamide hemifumarate (10 g) was dissolved in methanol (200 mL) at 25±5° C. The clear solution was filtered through HYFLO® to remove any undissolved particulate and subjected to spray drying in a laboratory spray dryer (Model Buchi-290) with feed rate of the solution 10 mL/min and inlet temperature at 70° C. to yield amorphous tenofovir alafenamide hemifumarate.

### Example 2: Preparation of a Premix of Amorphous Tenofovir Alafenamide Hemifumarate with 50% w/w PLASDONE S-630

Tenofovir alafenamide hemifumarate (5 g) was dissolved in methanol (100 mL) at 25±5° C. The resulting clear solution was filtered through HYFLO® to remove any undissolved particulate. In another flask, PLASDONE S-630 (5 g) was dissolved in methanol (100 mL) at 25±5° C. and this clear solution was added to tenofovir alafenamide hemifumarate solution at same temperature. The resulting clear solution was subjected to spray drying in a laboratory spray dryer (Model Buchi-290) with feed rate of the solution 10 mL/min and inlet temperature at 70° C. to yield a premix of amorphous tenofovir alafenamide hemifumarate.

### Example 3: Preparation of a Premix of Amorphous Tenofovir Alafenamide Hemifumarate with 33% w/w PLASDONE S-630

Tenofovir alafenamide hemifumarate (10 g) was dissolved in methanol (200 mL) at 25±5° C. The resulting clear solution was filtered through HYFLO® to remove any undissolved particulate. In another flask, PLASDONE S-630 (5 g) was dissolved in methanol (100 mL) at 25±5° C. and this clear solution was added to tenofovir alafenamide hemifumarate solution at same temperature. The resulting clear solution was subjected to spray drying in a laboratory spray dryer (Model Buchi-290) with feed rate of the solution 10 mL/min and inlet temperature at 70° C. to yield a premix of amorphous tenofovir alafenamide hemifumarate.

### Example 4: Preparation of a Premix of Amorphous Tenofovir Alafenamide Hemifumarate with 25% w/w PLASDONE S-630

Tenofovir alafenamide hemifumarate (10 g) was dissolved in methanol (200 mL) at 25±5° C. The resulting clear solution was filtered through HYFLO® to remove any undissolved particulate. In another flask, PLASDONE S-630 (2.5 g) was dissolved in methanol (50 mL) at 25±5° C. and this clear solution was added to tenofovir alafenamide hemifumarate solution at same temperature. The resulting clear solution was subjected to spray drying in a laboratory spray dryer (Model Buchi-290) with feed rate of the solution 10 mL/min and inlet temperature at 70° C. to yield a premix of amorphous tenofovir alafenamide hemifumarate.

### Example 5: Preparation of a Premix of Amorphous Tenofovir Alafenamide Hemifumarate with ˜40% w/w PLASDONE S-630

Tenofovir alafenamide hemifumarate (20 g) was dissolved in methanol (200 mL) at 25±5° C. The resulting clear solution was filtered through HYFLO® to remove any undissolved particulate. In another flask, PLASDONE S-630 (2.2 g) was dissolved in methanol (20 mL) at 25±5° C. and this clear solution was added to tenofovir alafenamide hemifumarate solution at same temperature. The resulting clear solution was subjected to spray drying in a laboratory spray dryer (Model Buchi-290) with feed rate of the solution 10 mL/min and inlet temperature at 70° C. to yield a premix of amorphous tenofovir alafenamide hemifumarate.

### Example 6: Preparation of a Premix of Amorphous Tenofovir Alafenamide Hemifumarate with 50% w/w Povidone K-30

Tenofovir alafenamide hemifumarate (10 g) was dissolved in methanol (200 mL) at 25±5° C. The resulting clear solution was filtered through HYFLO® to remove any undissolved particulate. In another flask, povidone K-30 (10 g) was dissolved in methanol (100 mL) at 25±5° C. and this clear solution was added to tenofovir alafenamide hemifumarate solution at same temperature. The resulting clear solution was subjected to spray drying in a laboratory spray dryer (Model Buchi-290) with feed rate of the solution 10 mL/min and inlet temperature at 70° C. to yield a premix of amorphous tenofovir alafenamide hemifumarate.

### Example 7: Preparation of a Premix of Amorphous Tenofovir Alafenamide Hemifumarate with 30 w/w % Povidone K-30

Tenofovir alafenamide hemifumarate (10 g) was dissolved in methanol (200 mL) at 25±5° C. The resulting clear solution was filtered through HYFLO® to remove any undissolved particulate. In another flask, povidone K-30 (5 g) was dissolved in methanol (100 mL) at 25±5° C. and this clear solution was added to tenofovir alafenamide hemifumarate solution at same temperature. The resulting clear solution was subjected to spray drying in a laboratory spray dryer (Model Buchi-290) with feed rate of the solution 10 mL/min and inlet temperature at 70° C. to yield a premix of amorphous tenofovir alafenamide hemifumarate.

### Example 8: Preparation of a Premix of Amorphous Tenofovir Alafenamide Hemifumarate with Povidone K-30 (25% w/w)

Tenofovir alafenamide hemifumarate (10 g) was dissolved in methanol (200 mL) at 25±5° C. The resulting clear solution was filtered through HYFLO® to remove any undissolved particulate. In another flask, povidone K-30 (2.5 g) was dissolved in methanol (50 mL) at 25±5° C. and this clear solution was added to tenofovir alafenamide hemifumarate solution at same temperature. The resulting clear solution was subjected to spray drying in a laboratory spray dryer (Model Buchi-290) with feed rate of the solution 10 mL/min and inlet temperature at 70° C. to yield a premix of amorphous tenofovir alafenamide hemifumarate.

### Example 9: Preparation of a Premix of Amorphous Tenofovir Alafenamide Hemifumarate with ˜40% w/w Povidone K-30

Tenofovir alafenamide hemifumarate (20 g) was dissolved in methanol (200 mL) at 25±5° C. The resulting clear solution was filtered through HYFLO® to remove any undissolved particulate. In another flask, povidone K-30 (2.2 g) was dissolved in methanol (50 mL) at 25±5° C. and this clear solution was added to tenofovir alafenamide hemifumarate solution at same temperature. The resulting clear solution was subjected to spray drying in a laboratory spray dryer (Model Buchi-290) with feed rate of the solution 10 mL/min and inlet temperature at 70° C. to yield a premix of amorphous tenofovir alafenamide hemifumarate.

### Example 10: Preparation of Premix of Amorphous Tenofovir Alafenamide Hemifumarate with ˜10% w/w β-Cyclodextrin

Tenofovir alafenamide hemifumarate (20 g) was dissolved in methanol (200 mL) at 25±5° C. The resulting clear solution was filtered through HYFLO® to remove any undissolved particulate. In another flask, O-cyclodextrin (2.2 g) was dissolved in water (180 mL) at 25±5° C. and this clear solution was added to tenofovir alafenamide hemifumarate solution at same temperature. The resulting clear solution was subjected to spray drying in a laboratory spray dryer (Model Buchi-290) with feed rate of the solution 10 mL/min and inlet temperature at 70° C. to yield a premix of amorphous tenofovir alafenamide hemifumarate.

### Example 11: Preparation of Premix of Amorphous Tenofovir Alafenamide Hemifumarate with ˜40% w/w Hydroxypropyl-β-Cyclodextrin

Tenofovir alafenamide hemifumarate (20 g) was dissolved in methanol (300 mL) at 25±5° C. The resulting clear solution was filtered through HYFLO® to remove any undissolved particulate. In another flask, hydroxypropyl-β-cyclodextrin (2.2 g) was dissolved in methanol (100 mL) at 25±5° C. and this clear solution was added to tenofovir alafenamide hemifumarate solution at same temperature. The resulting clear solution was subjected to spray drying in a laboratory spray dryer (Model Buchi-290) with feed rate of the solution 10 mL/min and inlet temperature at 70° C. to yield a premix of amorphous tenofovir alafenamide hemifumarate.

We claim:

1. A process for preparing a premix containing amorphous tenofovir alafenamide hemifumarate comprising:
a) dissolving tenofovir alafenamide hemifumarate in a solvent to form a solution;
b) combining the solution with a pharmaceutically acceptable excipient; and
c) removing the solvent to isolate the premix containing amorphous tenofovir alafenamide hemifumarate.

2. The process according to claim 1, wherein the solvent is selected from the group consisting of an alcohol solvent, a ketone solvent, a chlorinated solvent, water, and miscible mixtures thereof.

3. The process according to claim 2, wherein the solvent is selected from the group consisting of methanol, ethanol, propanol, isopropanol, n-butanol, sec-butanol, 2-butanol, t-butanol, 1-pentanol, 2-pentanol, 3-pentanol, 2-methyl-1-butanol, 2-methyl-1-butanol, 2,2-methyl-2-butanol, 3-methyl-2-butanol, 2,2-dimethyl-1-propanol, 1,1-dimethyl-1-propanol, and mixtures thereof.

4. The process according to claim 3, wherein the solvent is removed by evaporation, distillation, spray drying, lyophilization, or agitated thin film drying.

5. The process according to claim 1, wherein the pharmaceutical excipient is selected from the group consisting of polysaccharides, polyvinylpyrrolidone, polyvinyl acetate, polyvinyl alcohol, polymers of acrylic acid and salts thereof, polyacrylamide, polymethacrylates, vinylpyrrolidone-vinyl acetate copolymers, C₁-C₆ polyalkylene glycols, and mixtures thereof.

6. The process according to claim 5, wherein the polysaccharide is selected from the group consisting of hydroxypropyl methyl cellulose, croscarmellose, carboxymethyl cellulose, a sodium salt of carboxymethyl cellulose, a calcium salt of carboxymethyl cellulose, methyl cellulose, hydroxyethyl cellulose, ethyl hydroxyethyl cellulose, hydroxypropyl cellulose, microcrystalline cellulose, optionally substituted α-cyclodextrins, optionally substituted β-cyclodextrins, optionally substituted γ-cyclodextrins, and mixtures thereof.

7. The process according to claim 5, wherein the vinylpyrrolidone-vinyl acetate copolymer comprises N-vinyl-2-pyrrolidone and vinyl acetate in a 60:40 ratio, by mass.

8. The process according to claim 5, wherein the C₁-C₆ polyalkylene glycol is selected from the group consisting of polyethylene glycol, polypropylene glycol, and mixtures thereof.

9. The process according to claim 6, wherein said optionally substituted cyclodextrin is selected from the group consisting of β-cyclodextrin, hydroxypropyl-β-cyclodextrin, and mixtures thereof.

10. The process according to claim 1, further comprising dissolving the one or more pharmaceutical excipients in a second solvent to form a second solution.

11. The process according to claim 10, wherein the solvent and the second solvent are the same.

12. The process according to claim 3, wherein the solvent is methanol.

13. The process according to claim 5, wherein the excipient is polyvinylpyrrolidinone.

14. The process according to claim 1, wherein the excipient is copovidone.

15. The process according to claim 4, wherein the solvent is removed by evaporation.

16. The process according to claim 1, wherein the excipient is copovidone, the solvent is methanol, and the solvent is removed by evaporation.

17. The process according to claim 1, wherein the excipient is polyvinylpyrrolidinone, the solvent is methanol, and the solvent is removed by evaporation.
